SEQUENCE LISTING (3 sequences)

SEQ ID NO: 1 - protein, 602 aa, Bordetella bronchiseptica
DPNTVSIIKAGERQHGIHIKQSDGAGVRTATGTTIKVSGRQAQGVLLENPAAELRFQNGSVTSSGQLFDEGVRRFLGTVTVKAGKLVADHATLANVSDTRDDDGIALYVAGEQAQASIADSTLQGAGGVRVERGANVTVQRSTIVDGGLHIGTLQPLQPEDLPPSRVVLGDTSVTAVPASGAPAAVSVFGANELTVDGGHITGGRAAGVAAMDGAIVHLQRATIRRGDAPAGGAVPGGAVPGGFGPLLDGWYGVDVSDSTVDLAQSIVEAPQLGAAIRAGRGARVTVSGGSLSAPHGNVIETGGGARRFPPPASPLSITLRAGARAQGRALLYRVLPEPVKLTLAGGAQGQGDIVATELPPIPGASSGPLDVALASQARWTGATRAVDSLSIDNATWVMTDNSNVGALRLASDGSVDFQQPAEAGRFKVLMVDTLAGSGLFRMNVFADLGLSDKLVVMRDASGQHRLWVRNSGSEPASANTMLLVQTPRGSAATFTLANKDGKVDIGTYRYRLAANGNGQWSLVGAKAPPAPKPAPQPGPQPGPQPGPQPPQPPQPPQPPQRQPEAPAPQPPAGRELSAAANAAVNTGGVGLASTLWYAESN

SEQ ID NO: 2 - DNA, 1806 bp, Bordetella bronchiseptica
gatccaaacactgtgtcaatcatcaaggccggcgagcgccagcacggcatccacatcaagcaaagcgatggcgccggcgtacggaccgccaccggaacgaccatcaaggtaagcggtcgtcaggcccagggcgtcctgctggaaaatcccgcggccgagctgcggttccagaacggcagcgtcacgtcttcgggacagctgttcgacgaaggcgtccggcgctttctgggcaccgtcaccgtcaaggccggcaagctggtcgccgatcacgccacgctggccaacgtcagcgacacccgggacgacgacggcatcgcgctctatgtggccggcgagcaggcccaggccagcatcgccgacagcaccctgcagggcgcgggcggcgtgcgggtcgagcgcggcgccaatgtcacggtccaacgcagcaccatcgttgacgggggcttgcatatcggcaccctgcagccgctgcagccggaagaccttccgcccagccgggtggtgctgggcgacaccagcgtgaccgccgtgcccgccagcggcgcgcccgcggcggtgtctgtattcggggccaatgagcttacggttgatggcgggcacatcaccggggggcgggcagcgggggtggcggccatggacggggcgatcgtgcatctgcagcgcgcgacgatacggcggggggacgcgcctgccggcggtgcggttccaggcggtgcggttcccggcggcttcggccccctccttgacggctggtatggcgtggatgtatcggactccaccgtggacctcgctcagtcgatcgtcgaggcgccgcagctgggcgccgcgatccgggcgggccgcggcgccagggtgacggtgtcgggcggcagcttgtccgcaccgcacggcaatgtcatcgagaccggcggcggtgcgcgtcgcttcccgcctccggcctcgcccctgtcgatcaccttgcgggcgggcgcacgggcgcaggggagggcgctgctgtaccgggtcctgccggagcccgtgaagctgacgctggcgggcggcgcccaggggcagggcgacatcgtcgcgacggagctgcctcccattccaggcgcgtcgagcgggccgctcgacgtggcgctggccagccaggcccgatggacgggcgctacccgcgcggtcgactcgctgtccatcgacaacgccacctgggtcatgacggacaactcgaacgtcggcgcgctgcggctggccagcgacggcagcgtcgatttccagcagccggccgaagctgggcggttcaaggtcctgatggtcgatacgctggcgggttcggggctgttccgcatgaatgtcttcgcggacctggggctgagcgacaagctggtcgtcatgcgggacgccagcggccagcacaggctgtgggtccgcaacagcggcagcgagccggccagcgccaacaccatgctgctggtgcagacgccacgaggcagcgcggcgacctttacccttgccaacaaggacggcaaggtcgatatcggtacctaccgctatcgattggccgccaacggcaatgggcagtggagcctggtgggcgcgaaggcgccgccggcgcccaagcccgcgccgcagcccggtccccagcccggtccccagcccggtccccagccgccgcagccgccgcagccgccgcagccgccacagaggcagccggaagcgccggcgccgcaaccgccggcgggcagggagttgtccgccgccgccaacgcggcggtcaacacgggtggggtgggcctggccagcacgctctggtacgccgaaagcaat

SEQ ID NO: 3 - protein, 599 aa, Bordetella bronchiseptica
DWNNQSIIKAGERQHGIHIKQSDGAGVRTATGTTIKVSGRQAQGVLLENPAAELRFQNGSVTSSGQLFDEGVRRFLGTVTVKAGKLVADHATLANVSDTRDDDGIALYVAGEQAQASIADSTLQGAGGVRVERGANVTVQRSTIVDGGLHIGTLQPLQPEDLPPSRVVLGDTSVTAVPASGAPAAVSVFGANELTVDGGHITGGRAAGVAAMDGAIVHLQRATIRRGDAPAGGAVPGGAVPGGFGPLLDGWYGVDVSDSTVDLAQSIVEAPQLGAAIRAGRGARVTVSGGSLSAPHGNVIETGGGARRFPPPASPLSITLQAGARAQGRALLYRVLPEPVKLTLAGGAQGQGDIVATELPPIPGASSGPLDVALASQARWTGATRAVDSLSIDNATWVMTDNSNVGALRLASDGSVDFQQPAEAGRFKCLMVDTLAGSGLFRMNVAFADLGLSDKLVVMRDASGQHRLLVRNSGSEPASGNTMLLVQTPRGSAATFTLANKDGKVDIGTYRYRLAANGNGQWSLVGAKAPPAPKPAPQPGPQPGPQPPQPPQPPQPPQRQPEAPAPQPPAGRELSAAANAAVNTGGVGLASTLWYAESN

(19) United States
(12) Patent Application Publication
(10) Pub. No.: US 2006/0251674 A1
(43) Pub. Date: Nov. 9, 2006

(54) Formulations and process for production of Bordetella bronchiseptica P68 antigen and vaccines

(76) Inventors: Paul J. Dominowski, Hickory Corners, MI (US); Joseph C. Frantz, Nazareth, PA (US); Richard L. Krebs, Ashland, NE (US); Shelly L. Shields, Plainwell, MI (US); Robert Greg Sorensen, Lincoln, NE (US); Jeffrey E. Galvin, Mattawan, MI (US)

Correspondence Address: PHARMACIA & UPJOHN, 7000 Portage Road, KZO-300-104, KALAMAZOO, MI, 49001, US

(21) Appl. No.: 11/393,602

(22) Filed: Mar. 30, 2006

Related U.S. Application Data
(60) Provisional application No. 60/669,277, filed on Apr. 7, 2005.

(51) Int. Cl. A61K 39/295; A61K 39/116; A61K 39/10; A61K 48/00

(52) U.S. Cl. 424/201.1; 424/253.1; 514/44; 424/203.1

(57) ABSTRACT

The present invention comprises new formulations and a process for making such formulations for vaccine compositions comprising a Bordetella bronchiseptica p68 antigen.

### CROSS-REFERENCE TO RELATED APPLICATIONS

This application claims the benefit of U.S. Provisional Patent Application Ser. No. 60/669,277, filed Apr. 7, 2005 which is incorporated by reference within.

### BACKGROUND OF THE INVENTION

1. Field of Invention

This invention relates to new vaccine formulations comprising a Bordetella bronchiseptica p68 antigen, and a new process for making such formulations, and the use thereof for protecting dogs against infectious tracheobronchitis (“kennel cough”) caused by Bordetella bronchiseptica. The vaccines of the present invention provide increased immune response to vaccination and increased antibody titers to p68. Methods for protecting dogs against diseases caused by canine pathogens are also provided.

2. Background Art

The present commercially available canine Bordetella bronchiseptica vaccine product is composed of an inactivated, nonadjuvanted Bordetella bronchiseptica whole cell bacterin. Such whole cell bacterin can lead to cell protein related post-vaccination reactions. The p68 protein of B. bronchiseptica is antigenically similar to the Outer Membrane Protein (OMP) of B. pertussis and the OMP of B. parapertussis (Shahin et al., “Characterization of the Protective Capacity and Immunogenicity of the 69-kD Outer Membrane Protein of Bordetella pertussis”, J. Exp. Med., 171: 63-73, 1990). A protective role of this OMP has been demonstrated for mice (Shahin et al., supra; Novotny et al., “Biologic and Protective Properties of the 69-kD Outer Membrane Protein of Bordetella pertussis: A Novel Formulation for a Acellular Pertussis Vaccine”, J. Infect. Dis. 164:114-22, 1991), humans (He et al., “Protective Role of Immunoglobulin G Antibodies to Filamentous Hemagglutinin and Pertactin of Bordetella pertussis in Bordetella parapertussis Infection”, Eur. J. Clin Microbiol Infect Dis. 10:793-798, 1996) and swine (Kobisch et al., “Identification of a 68-Kilodalton Outer Membrane Protein as the Major Protective Antigen of Bordetella bronchiseptica by Using Specific-Pathogen-Free Piglets”, Infect. Immun. 58(2):352-357, 1990).

A prior vaccine composition comprising p68 antigen was shown to be effective in protecting canines against infectious tracheobronchitis (“kennel cough”) caused by Bordetella bronchiseptica. (See U.S. patent application Ser. No. 10/767,809) The application also relates to vaccines comprising the p68 antigen plus other canine pathogens. Some combination vaccines without the p68 antigen have been developed, including those sold under the Vanguard® tradename, and those disclosed in U.S. patent application Ser. No. 10/959,757.

The combination vaccines referred to above may include one or more antigens of other canine pathogens such as canine distemper (CD) virus, canine adenovirus type 1 (CAV-1), canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, Leptospira pomona, Leptospira hardjobovis, Leptospira hardjo, Porphyromonas spp., Bacteriodes spp., Leishmania spp., Borrelia spp., Ehrlichia spp., Mycoplasma ssp. and Microsporum canis.

CD is a universal, high-mortality viral disease with variable manifestations. Approximately 50% of nonvaccinated, nonimmune dogs infected with CD virus develop clinical signs, and approximately 90% of those dogs die.

Infectious canine hepatitis (ICH) caused by canine adenovirus type 1 (CAV-1), is a universal, sometimes fatal, viral disease of dogs characterized by hepatic and generalized endothelial lesions. Canine adenovirus type 2 (CAV-2) causes respiratory disease, which, in severe cases, may include pneumonia and bronchopneumonia.

CPI is a common viral upper respiratory disease. Uncomplicated CPI may be mild or sub-clinical, with signs becoming more severe if concurrent infection with other respiratory pathogens exists.

CPV infection results in enteric disease characterized by sudden onset of vomiting and diarrhea, often hemorrhagic. Leukopenia commonly accompanies clinical signs. Susceptible dogs of any age can be affected, but mortality is greatest in puppies. In puppies 4-12 weeks of age CPV may occasionally cause myocarditis that can result in acute heart failure after a brief and inconspicuous illness. Following infection many dogs are refractory to the disease for a year or more. Similarly, seropositive bitches may transfer to their puppies CPV antibodies, which can interfere with active immunization of the puppies through 16 weeks of age.

CCV also causes enteric disease in susceptible dogs of all ages worldwide. Highly contagious, the virus is transmitted primarily through direct contact with infectious feces, and may cause clinical enteritis within 1-4 days after exposure. Severity of disease may be exacerbated by concurrent infection with other agents. Primary signs of CCV infection include anorexia, vomiting, and diarrhea. Frequency of vomiting usually diminishes within a day or 2 after onset of diarrhea, but diarrhea may linger through the course of infection, and stools occasionally may contain streaks of blood. With CCV infection most dogs remain a febrile and leucopenia is not observed in uncomplicated cases.

Leptospirosis occurs in dogs of all, ages, with a wide range of clinical signs and chronic nephritis generally following acute infection.

The p68 vaccine compositions of the present invention, comprising a new formulation made by a new process, produce surprisingly elevated antibody titers to a p68 antigen, and are safe and effective in dogs.

### SUMMARY OF THE INVENTION

The present invention comprises a process for making vaccines and a formulation for vaccine compositions containing a Bordetella bronchiseptica p68 antigen, which result in surprisingly elevated antibody titers to p68, and effectively protect dogs against disease caused by Bordetella bronchiseptica. The vaccine compositions of the present invention do not cause significant post-vaccination reactions, are safe for administration to puppies, and induce protective immunity in dogs that lasts for an extended period of time.

In one aspect, the invention provides an antigen composition comprising a therapeutically effective amount of p68 protein and an amount of sodium dodecyl sulfate, wherein the amount of sodium dodecyl sulfate is from about 0.0005 percent to about 0.08 percent (w/v), or wherein the amount of sodium dodecyl sulfate is from about 0.001 percent to about 0.01 percent (w/v), or wherein the amount of sodium dodecyl sulfate is from about 0.0025 percent to about 0.0035 percent (w/v).

In another aspect, the present invention provides for an antigen composition wherein the p68 protein comprises a polypeptide selected from the group consisting of an amino acid sequence set forth in SEQ ID NO: 1; and an amino acid sequence that has at least 90% sequence identity and/or homology to the amino acid sequence set forth in SEQ ID NO: 1. In a further aspect, the p68 protein is produced from a polynucleotide sequence that encodes a p68 protein comprising an amino acid sequence set forth in SEQ ID NO: 1, or an amino acid sequence that has at least 90% sequence identity and/or homology to the amino acid sequence set forth in SEQ ID NO: 1. In yet another aspect, the p68 protein is produced from a polynucleotide sequence has a sequence of SEQ ID NO: 2, or a polynucleotide sequence that has at least 90% sequence identity and/or homology to the polynucleotide sequence set forth in SEQ ID NO: 2.

In an additional aspect, the invention provides for an antigen composition wherein the amount of p68 protein is about 2 to about 100 μg per dose, or wherein the amount of p68 protein is about 4 to about 45 μg per dose.

In another aspect, the invention provides for an antigen composition wherein the composition has a pH from about 9.5 to about 13, or wherein the antigen composition has a pH from about 10 to about 12.

In a further aspect, the invention provides for a vaccine composition comprising a carrier, more preferably wherein the carrier comprises saponin as a surfactant, and most preferably wherein the saponin is Quil A as the surfactant combined with cholesterol. The invention provides for a vaccine composition wherein the amount of Quil A is about 1 to about 100 μg per dose, and the amount of cholesterol is about 1 to about 100 μg per dose, or more preferably wherein the amount of Quil A is about 10 to about 50 μg per dose, and the amount of cholesterol is about 10 to about 50 μg per dose. The invention also provides for a vaccine composition wherein the carrier comprises aluminum hydroxide.

In yet another aspect of the invention, the vaccine composition has a pH from about 6 to about 9, or more preferably a pH from about 6.5 to about 8.0.

The present invention provides for a vaccine composition further comprising one or more antigens selected from the group consisting of canine distemper (CD) virus, canine adenovirus type 1 (CAV-1), canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, Leptospira pomona, Leptospira hardjobovis, and Leptospira hardjo.

Additionally, the invention provides to a vaccine composition wherein the amount of p68 protein is about 4 to about 45 μg per dose; the carrier is Quil A in an amount of about 10 to about 50 μg per dose and cholesterol in an amount of about 10 to about 50 μg per dose; the composition has a pH from about 6.5 to about 8.0; and the composition further comprises antigens of canine distemper (CD) virus, canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, and Leptospira pomona.

The invention provides for a method of protecting a canine from infection comprising the step of administering to the canine a therapeutically effective amount of a vaccine composition of the present invention.

Another important object of the present invention is a process for producing an antigen composition comprising the steps of suspending inclusion bodies containing p68 protein in a buffer solution having a pH from about 9.5 to about 13; and adding sodium dodecyl sulfate to a concentration of about 0.0005 percent to about 0.08 percent (w/v).

The present invention provides for a process of producing an antigen composition wherein the amount of sodium dodecyl sulfate is from about 0.001 percent to about 0.01 percent (w/v), or wherein the amount of sodium dodecyl sulfate is from about 0.0025 percent to about 0.0035 percent (w/v).

Another important object of the present invention provides for the use in the process of producing an antigen composition of a p68 protein comprising a polypeptide selected from the group consisting of an amino acid sequence set forth in SEQ ID NO: 1; and an amino acid sequence that has at least 90% sequence identity and/or homology to the amino acid sequence set forth in SEQ ID NO: 1. The invention also provides for the amount of p68 protein to be about 2 to about 100 μg per dose, or preferably to be about 4 to about 45 μg per dose.

Another important object of the invention is to provide a process further comprising the additional step, after adding the sodium dodecyl sulfate, of combining the antigen composition with a carrier, said carrier having a pH from about 6.5 to about 8.0. The invention further provides for the carrier to be Quil A and cholesterol.

An important object of the invention is to provide for a method of protecting a canine from infection comprising the step of administering to the canine a therapeutically effective amount of a composition produced by a process of this invention.

The present invention provides for a process of producing an antigen composition wherein the buffer solution is a carbonate buffer.

The present invention also provides for a process of an antigen composition further comprising a step of adding to the antigen composition one or more antigens selected from the group consisting of canine distemper (CD) virus, canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, Leptospira pomona, Leptospira hardjobovis, and Leptospira hardjo.

The present invention provides for a process of preparing an antigen composition further comprising a step of clarifying the composition by filtration or centrifugation. Additionally the invention provides for a process of preparing an antigen composition further comprising a step of sterilizing the composition. In the present invention, the composition may be sterilized by filtration.

Yet another important object of the present invention is a process of producing an antigen composition wherein a p68 protein is produced by steps comprising cloning into an expression vector a polynucleotide sequence that encodes a p68 protein comprising an amino acid sequence set forth in SEQ ID NO: 1, or an amino acid sequence that has at least 90% sequence identity and/or homology to the amino acid sequence set forth in SEQ ID NO: 1.; introducing the expression vector into a bacterial cell; and expressing the p68 protein, which accumulates in inclusion bodies. In yet a further important aspect of the present invention, the polynucleotide sequence has a sequence of SEQ ID NO: 2, or a polynucleotide sequence that has at least 90% sequence identity and/or homology to the polynucleotide sequence set forth in SEQ ID NO: 2. In another aspect of the present invention, the bacterial cell is Escherichia coli.

A further object of the present invention is to provide for a use of a novel composition of the present invention in the manufacture of a medicament for protecting dogs against diseases caused by canine pathogens.

These, and other aspects, will readily be apparent to those skilled in the art.

### DETAILED DESCRIPTION OF THE INVENTION

### Definitions

The term “inclusion bodies” refers to bodies formed within bacterial cells for the storage of various materials. Bacterial systems that express proteins within the cytoplasm form protein-filled inclusion bodies due to the aggregation of misfolded proteins. Inclusion bodies particularly form when cells are forced to express heterologous or mutant proteins, or when they over-express some endogenous proteins. The inclusion bodies generally contain extremely high concentrations of aggregated proteins. This suggests that the machinery for folding and/or processing proteins is saturated. For example, when bacterial cells are induced to express recombinant p68, the p68 is found in inclusion bodies in the cytoplasm of the cells.

The term “protecting a dog against a disease caused by a canine pathogen” as used herein means reducing or eliminating the risk of infection by the pathogen, ameliorating or alleviating the symptoms of an infection, or accelerating the recovery from an infection. Protection is achieved if there is a reduction in viral or bacterial load, a reduction in viral or bacterial shedding, a decrease in incidence or duration of infections, reduced acute phase serum protein levels, reduced rectal temperatures, and/or increase in food uptake and/or growth, for example.

The term “monovalent vaccine” as used herein refers to a vaccine having one principal antigenic component. For example, a p68 monovalent vaccine includes a Bordetella bronchiseptica p68 antigen as the principal antigenic component of the vaccine and is capable of protecting the animal to which the vaccine is administered against diseases caused by Bordetella bronchiseptica.

The term “combination vaccine” refers to a bivalent or multivalent combination of antigens, which are capable of inducing a protective immune response in an animal. The protective effects of a combination vaccine against a pathogen or pathogens are normally achieved by inducing in the animal subject an immune response, either a cell-mediated or a humoral immune response or a combination of both. For example, a p68 combination vaccine includes a Bordetella bronchiseptica p68 antigen in combination with one or more antigens of other canine pathogens as the principal antigenic components of the vaccine and is capable of protecting the animal to which the vaccine is administered against diseases caused by Bordetella bronchiseptica and the other pathogens.

The term “p68 vaccine” refers to both p68 monovalent and p68 combination vaccines.

By “immunogenic” is meant the capacity of a composition to provoke an immune response in animals against a particular pathogen. The immune response can be a cellular immune response mediated primarily by cytotoxic T-cells and cytokine-producing T-cells, or a humoral immune response mediated primarily by helper T-cells, which in turn activates B-cells leading to antibody production.

The term “therapeutically effective amount” or “effective amount” refers to an amount of a monovalent or combination vaccine sufficient to elicit a protective immune response in the animal to which it is administered. The immune response may comprise, without limitation, induction of cellular and/or humoral immunity. The amount of a vaccine that is therapeutically effective may vary depending on the particular antigen used in the vaccine, the age and condition of the animal, and/or the degree of infection, and can be determined by one skilled in the art.

For the purpose of the present invention, the term “p68 antigen” refers to a protein with a molecular weight of 68 kDa as determined by SDS polyacrylamide gel electrophoresis, is recognized by the p68-specific monoclonal antibody Bord 2-7 (ATCC# LN15898/PTA-5791), and has an amino acid sequence as set forth in SEQ ID NO: 1 or an amino acid sequence that is substantially identical to SEQ ID NO: 1. The term “p68 antigen” also includes a fragment of the protein that is recognized by this monoclonal antibody. By “substantially identical” is meant a degree of sequence identity of at least about 90%, preferably at least about 95%, or more preferably, at least about 98%. An example of a p68 antigen having an amino acid sequence substantially identical to SEQ ID NO: 1 is the p68 antigen described in WO 92/17587, which is set forth in SEQ ID NO: 3. The p68 specific monoclonal antibody of the present invention recognizes native p68 proteins, recombinant p68 proteins and p68 proteins on the surface of bacteria, for example.

The terms “carrier,” “acceptable carrier,” and “veterinary-acceptable carrier” includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers or stabilizing agents include albumin, among others.

The term “buffer” means a solution, suspension, emulsion, and the like comprising an ionic compound that resists changes in its pH. It includes, without limitation, carbonate, phosphate, TRIS, acetate, saline, and borate.

The term “C” when used in reference to temperature means centigrade or Celsius.

“Ambient temperature” is the air temperature surrounding an object. It is the temperature inside a room, which generally is from 15 to 25 degrees centigrade.

### Description of the Invention

In the following description of the invention, specific embodiments in which the invention may be practiced are described. These embodiments are described in sufficient detail to enable those skilled in the art to practice the invention. Other embodiments may be utilized, and logical and other changes may be made without departing from the scope of the invention. The following detailed description is, therefore, not to be taken in a limiting sense, and the scope of the invention is defined only by the appended claims, along with the full scope of equivalents to which such claims are entitled.

In accordance with the present invention, p68 antigens suitable for use in the present invention include both native p68 proteins (i.e., naturally occurring p68 proteins purified from Bordetella bronchiseptica) and recombinantly produced p68 proteins.

Recombinant production of p68 can be achieved using any one of the molecular cloning and recombinant expression techniques known to those skilled in the art. For example, a nucleic acid molecule encoding p68 can be introduced into an appropriate host cell, such as a bacterium, a yeast cell (e.g., a Pichia cell), an insect cell, or a mammalian cell (e.g., CHO cell). The p68-encoding nucleic acid molecule can be placed in an operable linkage to a promoter capable of effecting the expression of the p68 antigen in the host cell.

Purification of native p68 from Bordetella bronchiseptica is described, e.g., in Montaraz et al., Infection and Immunity 47: 744-751 (1985), and is also illustrated in previously referenced U.S. patent application Ser. No. 10/767,809. Recombinantly produced p68, which is expressed by the host cell, can be purified using routine protein purification techniques.

However, it is often difficult to obtain soluble, active proteins after expression in bacteria because of the production of inclusion bodies. While the inclusion bodies can easily be purified, the solubilization of the expressed protein usually can only be obtained using strongly denaturing conditions. It is then difficult to achieve efficient folding of the protein in-vitro while preventing aggregation. Proteins are prone to aggregation while the denaturing agent is being removed from the solution because they are not yet folded, and their hydrophobic regions, which were interacting with the denaturing agent, now interact with each other. Aggregation may be limited through the use of mild solubilizing agents during the refolding steps. However, most methods have produced only limited reduction in the amount of aggregated protein.

The inventors found that the present invention, comprising low levels of SDS in a buffer with a high pH, results in solubilized p68 protein that can be filtered and sterilized. This solubilized protein produces surprisingly elevated antibody titers to p68, effectively protecting dogs against disease caused by Bordetella bronchiseptica.

According to the present invention, either native p68 or recombinant p68 in inclusion bodies is solubilized in a buffer with a basic pH using a low concentration of sodium dodecyl sulfate (SDS) to form an antigen composition. The antigen composition is clarified and sterilized. In further accordance with the present invention, the antigen composition is combined with a veterinary-acceptable carrier having about a neutral pH to form a vaccine composition.

The amount of p68 in the vaccines should be immunizing-effective and is generally in the range of about 0.5 to about 1000 μg per dose. Preferably, the amount of p68 is in the range of about 1 to about 260 μg per dose. More preferably, the amount of p68 is in the range of about 2 to about 100 μg per dose. More preferably, the amount of p68 is about 4 to about 45 μg per dose.

The amount of sodium dodecyl sulfate used to solubilize the p68 protein is from about 0.0005 percent to about 0.08 percent (w/v), or more preferably from about 0.001 percent to about 0.01 percent (w/v), and most preferably from about 0.0025 percent to about 0.0035 percent (w/v).

Adjuvants suitable for use in accordance with the present invention include, but are not limited to, several adjuvant classes such as; mineral salts, e.g., Alum, aluminum hydroxide, aluminum phosphate and calcium phosphate; surface-active agents and microparticles, e.g., nonionic block polymer surfactants (e.g., cholesterol), virosomes, saponins (e.g., Quil A, QS-21 and GPI-0100), proteosomes, immune stimulating complexes, cochleates, quarterinary amines (dimethyl diocatadecyl ammonium bromide (DDA)), pyridine, vitamin A, vitamin E; bacterial products such as the RIBI adjuvant system (Ribi Inc.), cell wall skeleton of Mycobacterum phlei (Detox®), muramyl dipeptides (MDP) and tripeptides (MTP), monophosphoryl lipid A, Bacillus Calmete-Guerin, heat labile E. coli enterotoxins, cholera toxin, trehalose dimycolate, CpG oligodeoxnucleotides; cytokines and hormones, e.g., interleukins (IL-1, IL-2, IL-6, IL-12, IL-15, IL-18), granulocyte-macrophage colony stimulating factor, dehydroepiandrosterone, 1,25-dihydroxy vitamin D₃; polyanions, e.g., dextran; polyacrylics (e.g., polymethylmethacrylate, Carbopol 934P); carriers e.g., tetanus toxid, diptheria toxoid, cholera toxin B subnuit, mutant heat labile enterotoxin of enterotoxigenic E. coli (rmLT), heat shock proteins; oil-in-water emulsions e.g., AMPHIGEN® (Hydronics, USA); and water-in-oil emulsions such as, e.g., Freund's complete and incomplete adjuvants.

Preferred adjuvants for use in the vaccines of the present invention include Quil A and cholesterol (QAC). Another preferred adjuvant for use in the present invention includes aluminum hydroxide.

The amount of adjuvants suitable for use in the vaccines depends upon the nature of the adjuvant used. For example, when Quil A and cholesterol are used as adjuvant, Quil A is generally in an amount of about 1 to about 100 μg per dose, preferably 5 to about 75 μg per dose, and more preferably, about 10 to about 50 μg per dose. Cholesterol is generally in an amount of about 1 to about 100 μg per dose, preferably about 5 to about 75 μg per dose, and more preferably, about 10 to about 50 μg per dose. When aluminum hydroxide is used as adjuvant, it is generally in an amount of about 0.5 to about 20%, preferably about 0.5 to about 10%, and more preferably about 1 to about 2%.

The antigen composition and the acceptable carrier can be combined in any convenient and practical manner to form a vaccine composition, e.g., by admixture, solution, suspension, emulsification, encapsulation, absorption and the like, and can be made in formulations such as tablets, capsules, powder, syrup, solutions or suspensions that are suitable for injections, implantations, inhalations, ingestions or the like. Preferably, the vaccine is formulated such that it can be administered to dogs by injection in a dose of about 0.1 to about 5 ml, or preferably about 0.5 to about 2.5 ml, or even more preferably, in a dose of about 1 ml.

When appropriate, the pharmaceutical compositions of the present invention can be made sterile by well-known procedures. Sterilization of the media and reagents may be accomplished by heat sterilization or filter sterilization. Minimum heat sterilization requirements are about 121 degrees centigrade for about 30 minutes. Filter sterilization utilizes a filter with a maximum pore size of about 0.22 to about 0.3 microns. The vaccine compositions are generally filter-sterilized.

The pH of a solution may be adjusted using any appropriate acid or base, depending on the direction of adjustment needed. A preferred acid is citric acid; a preferred base is sodium hydroxide. During solubilization, the pH of the antigen composition is from about 8.5 to about 13.5, or more preferably from about 9.5 to about 13, or most preferably from about 10 to about 12. The final pH of the vaccine composition is from about 5 to about 9, or more preferably from about 6 to about 8, or most preferably from about 6.5 to about 8.0.

During solubilization of the p68, the antigen composition is incubated at ambient temperature from about 1 to about 10 hours, or preferably from about 1 to 5 hours, or more preferably from about 1 to about 2 hours.

In an embodiment of the present invention, the nucleotide sequence as set forth in SEQ ID NO: 2 coding for the p68 antigen that has the amino acid sequence of SEQ ID NO: 1, is cloned in an expression vector and placed in an operable linkage to a temperature sensitive promoter. The expression vector is introduced into Escherichia coli and the p68 antigen is expressed upon heat induction. The cells are lysed and the inclusion bodies where the p68 antigen accumulates are separated by centrifugation. The inclusion bodies are suspended in a carbonate buffer having a pH from about 9.5 to about 13. Advantageously, during solubilization the pH may be adjusted to about 12 with sodium hydroxide, and the solution incubated at ambient room temperature for about 2 hours. Following incubation, the pH is adjusted to about 10 with citric acid. Sodium dodecyl sulfate (SDS) is added to a concentration of about 0.003 percent (w/v). The solution is then clarified and sterilized by filtration to form an antigen composition. The antigen composition is then combined with a veterinary-acceptable carrier having a pH of about 7 to form a vaccine composition.

In another embodiment, the present invention provides methods of protecting dogs against disease caused by Bordetella bronchiseptica by administering to a dog a p68 vaccine composition, as described hereinabove. In accordance with the present invention, the p68 vaccine composition provides dogs with a long-term immunity for at least about 4 months, preferably for at least about 6 months, or even more preferably, for about one year or longer.

In accordance with the present invention, a p68 vaccine can be administered to a dog by any known routes, including the oral, intranasal, mucosal, topical, transdermal, and parenteral (e.g., intravenous, intraperitoneal, intradermal, subcutaneous or intramuscular). Administration can also be achieved using needle-free delivery devices. Administration can be achieved using a combination of routes, e.g., first administration using a parental route and subsequent administration using a mucosal route. Preferred routes of administration include subcutaneous and intramuscular routes.

The p68 vaccine compositions of the present invention can be administered to dogs of any age. Preferably, the dogs are from about 6 weeks to about 9 weeks old. Dogs can be vaccinated with one or more doses of a p68 vaccine, with about 2-4 weeks between each dose, preferably with about 3 weeks between doses. Preferably, two doses of a p68 vaccine are administered to dogs with an interval of about 2-4 weeks, preferably about 3 weeks, between the two administrations. If dogs are vaccinated before the age of 4 months, it is recommended that they be revaccinated with a single dose upon reaching about 4 months of age, because maternal antibodies may interfere with development of an adequate immune response in puppies less than 4 months old. Dogs can also be revaccinated annually with a single dose. Where B. bronchiseptica exposure is likely, such as breeding, boarding, and showing situations, an additional booster may be given within about 1 year, or preferably about 6 months, of the occurrence of these events.

Combination Vaccines

The combination vaccines of the present invention comprise a Bordetella bronchiseptica p68 antigen, which can be made as described hereinabove, in combination with at least one antigen from other canine pathogens capable of inducing a protective immune response in dogs against disease caused by such other pathogens. Such other pathogens include, but are not limited to, canine distemper (CD) virus, canine adenovirus type 1 (CAV-1), canine adenovirus type 2 (CAV-2), canine parainfluenza (CPI) virus, canine parvovirus (CPV), canine coronavirus (CCV), canine herpesvirus, and rabies virus. Antigens from these pathogens for use in the vaccine compositions of the present invention can be in the form of a modified live viral preparation or an inactivated viral preparation. Methods of attenuating virulent strains of these viruses and methods of making an inactivated viral preparation are known in the art and are described in, e.g., U.S. Pat. Nos. 4,567,042 and 4,567,043.

Other pathogens also include Leptospira bratislava, Leptospira canicola, Leptospira grippotyphosa, Leptospira icterohaemorrhagiae, Leptospira pomona, Leptospira hardjobovis, Leptospira hardjo, Porphyromonas spp., Bacteriodes spp., Leishmania spp., Borrelia spp., Ehrlichia spp., Mycoplasma ssp. and Microsporum canis. Antigens from these pathogens for use in the vaccine compositions of the present invention can be in the form of an inactivated whole or partial cell preparation, using methods well known in the art. For example, methods of making an inactivated whole or partial Leptospira cell preparation are known in the art and are described in, e.g., Yan, K-T, “Aspects of Immunity to Leptospira borgpetersenii serovar hardjo”, PhD Thesis, Appendix 1,1996. Faculty of Agriculture and Food Science, The Queen's University of Belfast; Mackintosh et al., “The use of a hardjo-pomona vaccine to prevent leptospiruria in cattle exposed to natural challenge with Leptospia interrogans serovar hardjo”, New Zealand Vet. J. 28:174-177, 1980; Bolin et. al., “Effect of vaccination with a pentavalent leptopsiral vaccine on Leptospira interrogans serovar hardjo type hardjo-bovis infection of pregnant cattle”, Am. J. Vet. Res. 50:161-165, 1989.

Combination vaccines may prepared by rehydrating a freeze-dried preparation of the attenuated viral strains (or a preparation made by other methods such as spray drying or desiccation) and viral preparation with a liquid preparation, which liquid preparation comprises the Leptospira and p68 antigens, dissolved in sterile saline solution and adjuvanted with Quil A and cholesterol. Such combination vaccine may also be prepared by rehydrating a freeze-dried preparation of the attenuated viral strains and Leptospira antigen preparation (or a preparation made by other methods such as spray drying or desiccation) with a sterile solution and adjuvanted with Quil A and cholesterol, or rehydrating said freeze-dried preparation with CCV, p68 plus diluent and adjuvanted with Quil A and cholesterol. The p68 antigen is solubilized according to the process of the present invention, using sodium dodecyl sulfate in an amount from about 0.0005 percent to about 0.08 percent (w/v), or more preferably from about 0.001 percent to about 0.01 percent (w/v), and most preferably from about 0.0025 percent to about 0.0035 percent (w/v).

In accordance with the present invention, combination vaccines can be administered to dogs of any age. Preferably, the dogs are from about 6 weeks to about 9 weeks old. The combination vaccines can be administered in 2 to 4 doses, preferably in 2 to 3 doses. The doses can be administered with about 2 to about 6 weeks between each dose, preferably with about 2 to about 4 weeks between each dose, and most preferably with about 3 weeks between each dose.

Preferred Combination Vaccine.

A preferred combination vaccine includes the attenuated CD virus strain designated as the “Snyder Hill” strain (National Veterinary Service Laboratory, Ames, Iowa), the attenuated CAV-2 strain designated as the “Manhattan” strain (National Veterinary Service Laboratory, Ames, Iowa), the attenuated CPI virus strain having the designation of “NL-CPI-5” (National Veterinary Service Laboratory, Ames, Iowa), the attenuated CPV strain having the designation of “NL-35-D” (National Veterinary Service Laboratory, Ames, Iowa), an inactivated preparation of the CCV strain having the designation of “NL-18” (National Veterinary Service Laboratory, Ames, Iowa), and the recombinant Bordetella bronchiseptica p68 antigen having the sequence of SEQ ID NO: 1. Such combination vaccine also includes inactivated whole cell preparations of five Leptospira species: Leptospira canicola (e.g., strain C-5, National Veterinary Service Laboratory, Ames, Iowa), Leptospira grippotyphosa (e.g., strain MAL 1540, National Veterinary Service Laboratory, Ames, Iowa.), Leptospira icterohaemorrhagiae (e.g., strain NADL 11403, National Veterinary Service Laboratory, Ames, Iowa), Leptospira bratislava (e.g., strain JEZ, National Veterinary Service Laboratory, Ames, Iowa) and Leptospira pomona (e.g., strain T262, National Veterinary Service Laboratory, Ames, Iowa). Such combination vaccine is preferably prepared by rehydrating a freeze-dried preparation of the attenuated viral strains (or a preparation made by other methods such as spray drying or desiccation) and viral preparation with a liquid preparation, which liquid preparation comprises the p68 antigen and Leptospiral antigens, dissolved in sterile saline solution and adjuvanted with Quil A and cholesterol. The concentration of sodium dodecyl sulfate used to solubilize the p68 protein is preferably from about 0.0025 percent to about 0.0035 percent (w/v).

The invention is described in greater detail by the following non-limiting examples.

### EXAMPLES

### Example 1

Large-scale quantities of Bordetella Bronchiseptica Bacterial Extract, Subunit (p68) were produced in the following manner.

I. Composition of the Product.

Microorganisms Used. A recombinant strain of Escherichia coli (LWP68), constructed to express outer membrane protein p68 from Bordetella bronchiseptica, was prepared.

A single colony of this construct was subcultured on Luria-Bertani agar supplemented with 50 mg/L of kanamycin sulfate (LB-KAN). A single colony of this culture was then subcultured to LB-KAN broth medium. Growth from this subculture was combined with cryopreservative and stored at −70° C. Pre-Master Seed was made from this material on May 9, 1994, and designated: Master Seed Bordetella bronchiseptica Extract Vaccine, Lot Number 001. Master Seed was prepared from this Pre-Master Seed and designated as Master Seed LWP68, E. coli/Bordetella bronchiseptica Recombinant p68, Lot Number 002.

The bacterium contains a plasmid insert, which provides resistance to inhibition of growth by kanamycin. The bacterium also carries a plasmid insert containing a gene coding for the p68 protein of Bordetella bronchiseptica. Expression of the p68 protein can be observed following heat induction of the culture when assayed by immunoblot using a p68-specific antibody.

II. Cultures.

1. Composition and Reaction of Media Used for Production Cultures Minimum heat sterilization requirements for media and reagents are 121° C., 30 minutes. Filter sterilization for media and reagents utilizes a filter with a maximum pore size of 0.22μ.

P68 Base Medium

| | | |
|---|---|---|
| Yeast extract | 13.35 | gm |
| Tryptone | 6.67 | gm |
| Glycerol | 0.00695 | L |
| Purified water q.s. | 1 | L |

Dissolve at 40-60° C., sterilize by heat or filtration.

P68 Salts

| | | |
|---|---|---|
| K2HPO4, 3H2O | 10 | gm |
| KH2PO4 | 5 | gm |
| Purified water q.s. | 1 | L |

Sterilize by heat or filtration.

Kanamycin Sulfate Solution

| | | |
|---|---|---|
| Kanamycin sulfate | 50 | gm |
| Purified water q.s. | 1 | L |

Sterilize by filtration.

Feed Medium Supplement

| | | |
|---|---|---|
| Glycerol | 500 | gm |
| Yeast extract | 90 | gm |
| Purified water q.s. | 1 | L |

Sterilize by heat or filtration.

2. Character, Size, and Shape of Container Used for Growing Cultures

| Type of Container | Size of Container | |
|---|---|---|
| Flasks | 1000-6000 | mL |
| Seed Fermentors | 40-600 | L |
| (optional) | | |
| Production Fermentors | 500-10,000 | L |

3. Storage Conditions of the Seed Cultures

| Master Seed or Master Cell Stock | Target Storage Temperature |
|---|---|
| Master Seed Bacteria (Frozen) | −60° C. or below |
| Master Seed Bacteria (Lyophilized) | 2-7° C. |
| Working Seed Bacteria (Frozen) | −30° C. or below |
| Working Seed Bacteria (Lyophilized) | 2-7° C. |

4. Methods of Preparing Suspensions for Seeding or Inoculation Working Seed cultures are prepared from flask cultures containing p68 complete medium and inoculated with Master Seed or Working Seed. The cultures may be stabilized with glycerol and aliquoted into sterile vials and frozen.

5. Technique of Inoculating Seed and Production Media

| | | | Volume of | | |
|---|---|---|---|---|---|
| Type of Container | Size of Container | | Medium | | % Inoculum |
| Flasks | 1000-6000 | mL | 300-5000 | mL | 0.001-2.0% |
| Seed Fermentors | 40-600 | L | 16-510 | L | 0.5-5.0% |
| (optional) | | | | | |
| Production | 500-10,000 | L | 200-9200 | L | 0.1-5.0% |
| Fermentors | | | | | |

Master or Working Seed is quickly thawed and used to inoculate flasks. Flasks are used as inoculum for seed or production fermentors.

6. Period of Time and Conditions for Incubation All cultures are incubated at 30±2° C.

| Stage | pH ¹\textcircled{1} | Conditions \textcircled{2}² | Incubation |
|---|---|---|---|
| Flask | Not controlled | Not controlled | 4-12 hours |
| Seed Fermentor | 7.0 ± 0.3 | Air/Agitation | 2-12 hours |
| (optional) | | | |
| Production Fermentor | 7.0 ± 0.3 | Air/Agitation | 8-24 hours ³\textcircled{3} |

¹Using 1.4-14 N ammonium hydroxide.
²Aeration and agitation are controlled to maintain an aerobic environment.
³Total incubation time at 30 ± 2° C. See below for heat induction.
Feed medium supplement is added to the culture as needed.

Production Fermentor At an OD of 10-50 (600 nm), the culture is induced to express p68 protein by rapidly raising the temperature to a minimum of 39° C. and maintaining a temperature of 39±2° C. for 2-4 hours.
7. Character and Amount of Growth Prior to harvest, the culture is examined microscopically for purity, characteristic morphology, and Gram Reaction. Growth is monitored by periodic optical density readings at 600 nm.
III. Harvest
A. Handling and Preparation of Cultures Prior to Harvest

At the end of the production cycle, cultures are examined by microscopy for characteristic morphology and Gram stained preparations are examined for the presence of contamination and to confirm that the bacteria are Gram negative.

B. Minimum and Maximum Times for Harvest

| Antigen | Minimum | Maximum |
|---|---|---|
| p68 | 8 hr | 24 hr |

C. Technique of Harvesting for Production Purposes

After determining the cell density, agitation is slowed, temperature is lowered to <20° C. and aeration and pH control are discontinued. The cells are separated from the culture fluid by centrifugation or microfiltration. The supernatant is discarded and the cells are resuspended in a lysate buffer. The inclusion bodies are released from the cells by physical disruption in a homogenizer. The fluids are centrifuged (or microfiltered) to separate and concentrate the inclusion bodies. The inclusion bodies are solubilized at high pH in carbonate buffer. Sodium dodecyl sulfate (SDS) is added and the fluids are sterile filtered.

D. Specifications for Acceptable Harvest Material

The culture must be free of contamination at the end of the induction period as described in Section II.7.

E. Additional Information

Solubilization is performed between pH 9.5 to 12.9 using NaOH and citric acid. When solubilization is complete, a solution of SDS is added to a final concentration of 0.003±0.0005% (w/v).

IV. Preparation of the Product

A. Composition of Adjuvant and Proportions Used.

50 mcg/ml of Quil A and 50 mcg/ml of cholesterol are added as an adjuvant to the product as described in Section IV.D.

B. Method and Degree of Concentration

Concentration of E. coli by centrifugation or microfiltration. Lysis of cells by homogenization. Concentration of inclusion bodies will be done by centrifugation or microfiltration.

C. Standardization of the Product

Fluids are assayed for p68 concentration by quantitative SDS-PAGE. The level of SDS per mL is calculated based on the amount added in Section III.C. Based on these results, the serial is standardized so that each dose contains ≦0.003% (w/v) of sodium dodecyl sulfate (calculated) and the following calculated antigen level/dose:

| | Minimum Standardization | Maximum Standardization |
|---|---|---|
| Antigen | Requirement | Requirement |
| p68 | 15 μg | 45 μg |

D. Assembly of Units to Prepare a Serial of Vaccine

For assembly of a serial, one or more complete or partial lots of p68 are combined with Quil A and saline as the aqueous phase. Following thorough mixing of the aqueous phase, cholesterol is slowly added to the aqueous phase with continuous homogenization to emulsify the serial.

Example Serial:

| | Bulk Antigen | Assembled | |
|---|---|---|---|
| Component | (RU/mL) | Antigen (RU/mL) | Volume (L) |
| P68 bulk fluid | 1,500 μg/mL | 40 μg | 8.0 |
| Quil A (50 mg/mL) | NA | NA | 0.30 |
| Cholesterol | NA | NA | 0.840 |
| (18 mg/mL) in | | | |
| 100% Ethanol | | | |
| Saline | NA | NA | 290.86 |
| Total Volume | | | 300 |

Based on a fill volume of 1.0 mL/dose, this 300,000 dose final product would have the following assembled antigen level per dose:

| Antigen | Final Product: Assembled Antigen Level/Dose |
|---|---|
| p68 | 40 μg |

### Example 2

This study compared and evaluated the safety of a p68 monovalent vaccine in combination with other products in minimum-age non-beagle dogs. The systemic and local tissue reactions to the vaccines, in puppies when vaccinated at approximately 6 weeks of age, were evaluated. The p68 monovalent vaccine was made according to the process of Example 1.

Materials and Method.

Animals. Commercially raised toy breed dogs of either gender were used in this study. Breeds included Maltese, Bichon Frise, Yorkshire Terrier, Pug, Dachsund, West Highland White Terrier, and Shih Tzu. At the time of vaccination dogs were 6+/−1 week of age. Animals were housed in pens by litter and observed at least once daily for mortality and morbidity in addition to study observations.

Study Design. The study design was a generalized randomized block design (GRBD). Litter was the blocking factor. Animal was the experimental unit. Treatments are summarized below.

| Treatment | | | Animals/ |
|---|---|---|---|
| Group | Diluent | Plug | Group |
| T01 | 25 mcg p68/50 mcg QAC | DA2PPCCV | 10 |
| | combined with | | |
| | 5 Leptospira serovars | | |
| T02 | 2 × 25 mcg p68/50 mcg | 2 × DA2PPCCV | 10 |
| | QAC combined | | |
| | with 5 Leptospira serovars | | |
| T03 | Duramune ® Max 5- | Duramune ® Max | 10 |
| | CvK/4L diluent | 5-CvK/4L | |

Vaccines.

Diluent
1. 25 mcg p68 antigen in 1 ml sterile Hal's buffer containing 50 mcg Quil A and 50 mcg cholesterol as an adjuvant (Serial # 59433-12) (True Name: Bordetella Bronchiseptica Bacterial Extract, Subunit, Code 2B05.R0), combined with five serovars of Leptospira (L. canicola, L. grippotyphosa, L. bratislava, L. icterhemorrhagiae, L. pomona), inactivated and reconstituted in Hal's buffer.
2. Commercially available Duramune® Max 5-CvK/4 L diluent (Serial # 094132A).

Plug
1. The experimental serial 311002-B, comprised of the following modified live viral components: Canine Distemper Virus, Canine Adeonovirus-2, Canine Parvovirus, Canine Parainfluenza Virus, and Canine Coronavirus, adjuvanted with 25 mcg QAC (True Name: Canine Distemper-Adenovirus Type 2-Coronavirus-Parainfluenza-Parvovirus Vaccine, Modified Live and Killed Virus, Code 1597.20).
2. Commercially available Duramune® Max 5-CvK/4 L (Canine Distemper-Adenovirus Type 2-Coronavirus-Parainfluenza-Parvovirus Vaccine, Modified Live and Killed Virus-Leptospira Bacterin, Fort Dodge Animal Health) (Serial # 116432A).

Challenge. No experimental challenge was performed in this study.

Pertinent Variables Measured.

Systemic Reaction Scores. Animals were observed for systemic changes prior to vaccination, five hours post-vaccination and then daily for a total of 9 days. The activity level was assessed and scored. Animals were given a score of “1” if their activity level was appropriate for the animal's age and environment. Animals were given a score of “2” if they were lethargic and reluctant to engage in play activity, and were given a score of “3” if they were moribund or resistant to move without significant coercion. If a score of “2” or “3” was given, a comment was included describing the animal's activity level.

Local Reaction Scores. Animals were examined prior to vaccination to determine if any pre-existing lesions were present at the potential injection site. Following vaccination (five hours following, and then daily for 14 days), injection sites were assessed for reactions. If a reaction was present and a measurement could be taken it was recorded. It was also noted if the injection site reaction appeared to be painful to the animal as well as any other comments relevant to the reaction. Animals were given a score of “1” if no palpable swelling or heat was present, a score of “2” if a small (less than 1 inch in largest measurement), non-painful swelling was present, and a score of “3” if the swelling was larger than 1 inch in any measurement, warm, or painful. Scores of “2” or “3” were accompanied by a description or measurement of the lesion.

Injection Site Reaction Volumes. Measurements obtained for local reaction scores were used to determine the reaction site volume.

Body Temperature. Body temperature was determined and recorded prior to vaccination and at each observation point following vaccination until Day 2.

Data Summary And Analysis.

Injection Site Reaction Volumes. Injection site volumes were calculated using the following formula: volume=length×height×thickness. If the local reaction score was 1, then a volume of 0 was used. The volumes were analyzed using a general linear mixed model for repeated measures with a model that included the fixed effects of treatment, observation time, and treatment by observation time interaction. If the treatment or treatment by observation time interaction was significant, then pairwise treatment comparisons were made at each observation time. If the observation time or treatment by observation time interaction was significant, then comparisons between the first observation time and subsequent observation times were made within each treatment.

Systemic and Local Reactions. Frequency distributions of systemic and local reactions were calculated for each treatment and observation time.

Rectal Temperatures. Descriptive statistics including the number of animals, arithmetic mean, standard error, minimum and maximum were calculated for each treatment at each time point temperatures were recorded.

All hypothesis tests were conducted at the 0.05 level of significance (P≦0.05).

Results.

No animals received a systemic reaction score of “3” during the study. However, two animals were determined to be systemically abnormal at the observation period 5 hours following vaccination, and therefore received a score of “2”. These consisted of one dog in group T01 and another in group T03 that were judged to be lethargic. The number of systemic reaction scores for each treatment and time point are summarized in Table 1.

TABLE 1
Systemic reaction scores

| | | Systemic Reaction Score | | |
|---|---|---|---|---|
| | | 1 | 2 | 3 |
| Day of Study | Treatment | Number of Animals | | |
| 0.0 | T01 | 10 | 0 | 0 |
| | T02 | 10 | 0 | 0 |
| | T03 | 10 | 0 | 0 |
| 0.2 | T01 | 9 | 1 | 0 |
| | T02 | 10 | 0 | 0 |
| | T03 | 9 | 1 | 0 |
| 1 | T01 | 10 | 0 | 0 |
| | T02 | 10 | 0 | 0 |
| | T03 | 10 | 0 | 0 |
| 2 | T01 | 10 | 0 | 0 |
| | T02 | 10 | 0 | 0 |
| | T03 | 10 | 0 | 0 |
| 3 | T01 | 10 | 0 | 0 |
| | T02 | 10 | 0 | 0 |
| | T03 | 10 | 0 | 0 |
| 4 | T01 | 10 | 0 | 0 |
| | T02 | 10 | 0 | 0 |
| | T03 | 10 | 0 | 0 |
| 5 | T01 | 10 | 0 | 0 |
| | T02 | 10 | 0 | 0 |
| | T03 | 10 | 0 | 0 |
| 6 | T01 | 10 | 0 | 0 |
| | T02 | 10 | 0 | 0 |
| | T03 | 10 | 0 | 0 |
| 7 | T01 | 10 | 0 | 0 |
| | T02 | 10 | 0 | 0 |
| | T03 | 10 | 0 | 0 |
| 8 | T01 | 10 | 0 | 0 |
| | T02 | 10 | 0 | 0 |
| | T03 | 10 | 0 | 0 |
| 9 | T01 | 10 | 0 | 0 |
| | T02 | 10 | 0 | 0 |
| | T03 | 10 | 0 | 0 |

Several animals in each treatment group were observed to have local reaction scores of “2” and “3”. The majority of these animals had non-painful areas of various sizes, which persisted for variable periods, with 2 animals in group T01 and 5 animals in group T02 persisting greater than two weeks. No animal received a Local Reaction Score of “3” after day 2. Number of local reaction scores for each treatment and time point are presented in Table 2.

TABLE 2
Local reaction scores

| | | Local Reaction Score | | |
|---|---|---|---|---|
| | | 1 | 2 | 3 |
| Day of Study | Treatment | Number of Animals | | |
| 0.0 | T01 | 10 | 0 | 0 |
| | T02 | 10 | 0 | 0 |
| | T03 | 10 | 0 | 0 |
| 0.2 | T01 | 1 | 4 | 5 |
| | T02 | 0 | 4 | 6 |
| | T03 | 1 | 6 | 3 |
| 1 | T01 | 0 | 6 | 4 |
| | T02 | 0 | 6 | 4 |
| | T03 | 1 | 4 | 5 |
| 2 | T01 | 2 | 8 | 0 |
| | T02 | 0 | 8 | 2 |
| | T03 | 2 | 8 | 0 |
| 3 | T01 | 3 | 7 | 0 |
| | T02 | 0 | 10 | 0 |
| | T03 | 7 | 3 | 0 |
| 4 | T01 | 4 | 6 | 0 |
| | T02 | 0 | 10 | 0 |
| | T03 | 8 | 2 | 0 |
| 5 | T01 | 3 | 7 | 0 |
| | T02 | 0 | 10 | 0 |
| | T03 | 10 | 0 | 0 |
| 6 | T01 | 3 | 7 | 0 |
| | T02 | 0 | 10 | 0 |
| | T03 | 10 | 0 | 0 |
| 7 | T01 | 2 | 8 | 0 |
| | T02 | 0 | 10 | 0 |
| | T03 | 10 | 0 | 0 |
| 8 | T01 | 3 | 7 | 0 |
| | T02 | 0 | 10 | 0 |
| | T03 | 10 | 0 | 0 |
| 9 | T01 | 4 | 6 | 0 |
| | T02 | 0 | 10 | 0 |
| | T03 | 10 | 0 | 0 |
| 10 | T01 | 4 | 6 | 0 |
| | T02 | 1 | 9 | 0 |
| | T03 | 10 | 0 | 0 |
| 11 | T01 | 6 | 4 | 0 |
| | T02 | 1 | 9 | 0 |
| | T03 | 10 | 0 | 0 |
| 12 | T01 | 5 | 5 | 0 |
| | T02 | 2 | 8 | 0 |
| | T03 | 10 | 0 | 0 |
| 13 | T01 | 5 | 5 | 0 |
| | T02 | 4 | 6 | 0 |
| | T03 | 10 | 0 | 0 |
| 14 | T01 | 8 | 2 | 0 |
| | T02 | 5 | 5 | 0 |
| | T03 | 10 | 0 | 0 |

Several differences in injection site volumes were noticed. On Days 0.2, 1 and 2, T01 and T02 were different than T03. On Day 2, T01 was also different than T03. On Day 3, T02 was different compared to T03, and this was again the case on Day 5. Injection site volumes greater than 0 (measurements are in inches³) are listed in Table 3.

TABLE 3
Injection site volumes

| Day of | | | |
|---|---|---|---|
| Study | T01 | T02 | T03 |
| 0.2 | 0.375, 0.391, 0.250, | 0.188, 0.250, 0.281, | 0.125, 0.031, 0.125, |
| | 0.156, 0.063, 0.250, | 0.125, 0.781, 0.156, | 0.063, 0.047, 0.125, |
| | 0.391, 0.250, 0.250 | 0.250, 0.125, 0.125, | 0.031 |
| | | 0.031 | |
| 1 | 0.125, 0.141, 0.188, | 0.375, 0.125, 0.188, | 0.125, 0.016, 0.031, |
| | 0.188, 0.125, 0.031, | 0.250, 0.094, 0.125, | 0.250, 0.031, 0.250, |
| | 0.063, 0.188, 0.125, | 0.063, 0.250, 0.031, | 0.016, 0.141, 0.031 |
| | 0.125 | 0.250 | |
| 2 | 0.125, 0.063, 0.063, | 0.250, 0.016, 0.125, | 0.047 |
| | 0.047, 0.031, 0.070, | 0.125, 0.125, 0.125, | |
| | 0.125, 0.125 | 0.188, 0.141, 0.250 | |
| 3 | 0.016, 0.125, 0.008, | 0.031, 0.047, 0.047, | 0.016 |
| | 0.047, 0.031, 0.023 | 0.047, 0.031, 0.023, | |
| | | 0.023, 0.125, 0.063 | |
| 4 | 0.016, 0.125, 0.016, | 0.023, 0.016, 0.094, | 0.008, 0.008 |
| | 0.016, 0.008, 0.008 | 0.047, 0.094, 0.016, | |
| | | 0.023, 0.031, 0.047 | |
| 5 | 0.016, 0.094, 0.012, | 0.016, 0.031, 0.023, | |
| | 0.004, 0.016, 0.016, | 0.125, 0.125, 0.125, | |
| | 0.016 | 0.031, 0.016, 0.063, | |
| | | 0.031 | |
| 6 | 0.008, 0.047, 0.012, | 0.008, 0.008, 0.012, | |
| | 0.008, 0.012, 0.008, | 0.023, 0.016, 0.035, | |
| | 0.016 | 0.016, 0.008, 0.016, | |
| | | 0.031 | |
| 7 | 0.008, 0.008, 0.008, | 0.004, 0.006, 0.016, | |
| | 0.004, 0.008, 0.004, | 0.008, 0.008, 0.016, | |
| | 0.006, 0.008 | 0.008, 0.012, 0.016, | |
| | | 0.031 | |
| 8 | 0.008, 0.008, 0.004, | 0.004, 0.004, 0.006, | |
| | 0.004, 0.004, 0.004, | 0.016, 0.006, 0.016, | |
| | 0.004 | 0.008, 0.004, 0.008, | |
| | | 0.031 | |
| 9 | 0.004, 0.004, 0.004, | 0.004, 0.002, 0.006, | |
| | 0.001, 0.004, 0.004 | 0.008, 0.004, 0.006, | |
| | | 0.004, 0.008, 0.004, | |
| | | 0.016 | |
| 10 | 0.004, 0.008, 0.004, | 0.004, 0.004, 0.002, | |
| | 0.001, 0.001, 0.004 | 0.004, 0.006, 0.002, | |
| | | 0.006, 0.004, 0.012 | |
| 11 | 0.004, 0.008, 0.001, | 0.001, 0.004, 0.008, | |
| | 0.004 | 0.008, 0.012, 0.004, | |
| | | 0.006, 0.004, 0.006 | |
| 12 | 0.008, 0.008, 0.001, | 0.004, 0.004, 0.002, | |
| | 0.002, 0.002 | 0.002, 0.006, 0.006, | |
| | | 0.002, 0.012 | |
| 13 | 0.004, 0.008, 0.001, | 0.004, 0.001, 0.001, | |
| | 0.002, 0.002 | 0.008, 0.001, 0.004 | |
| 14 | 0.004, 0.002 | 0.002, 0.001, 0.002, | |
| | | 0.002, 0.004 | |

Rectal temperatures usually remained acceptable (<103.5° F.) throughout the observation period, Day 0-Day 2. One animal in T01 and two animals in T02 had rectal temperatures greater than 103.5° at the five hours post-vaccination observation point (Day 0.2), and a third animal in T02 had an elevated rectal temperature at the Day 1 observation point.

2. Discussion

Vaccine reactions in companion animals are historically difficult to gauge and put into context. The intent of this study was to measure vaccine reactions in a population of animals, which represents those at the greatest risk for both systemic and local reactions. Following vaccination, two animals (one in the group that received a 1× dose of the vaccine under development and one in the group that received the commercially available vaccine) appeared lethargic and slightly obtunded. There is no concern with this degree of systemic reaction.

To evaluate local reactions, measurements were taken and differences were noted between treatment groups. However, the largest reaction size measured was 0.375 cubic inches in the 2× treatment group on the day following vaccination. All other reaction sizes were 0.25 cubic inches or less and after day 2 following vaccination, no injection site was measured to be greater than ⅛ of a cubic inch in volume. While this degree of tissue swelling is palpable by a skilled investigator, it is probably not noticeable to the average pet owner or companion animal veterinarian. Additionally, by repeatedly palpating the area, it is reasonable to assume that the injection site was irritated and any tissue reaction may have been prolonged by the act of measuring it.

Conclusion.

The p68 vaccine in combination with multiple antigens demonstrated clinically acceptable safety parameters.

### Example 3

This study was conducted to characterize the serological response to two vaccine formulations in dogs, and to assure that the process changes made no material difference in the antigenicity of the vaccine compositions. One vaccine formulation was made by the process of the current invention (Example 1); this is referred to as the “new process” in this example. The second formulation was made by the same process that was used to make the vaccine compositions used in U.S. patent application Ser. No. 10/767,809; this is referred to as the “old process” in this example. The vaccine compositions used in the 10/767,809 application were made using a solubilization process comprising the use of about 0.1% (w/v) sodium dodecyl sulfate.

Materials and Methods

Animals. Ten mixed-breed dogs that were between seven and eight months of age on Day 0 and of mixed sex arrived at the study location on Day-4. Animals were selected from a pool of acceptable animals provided by a commercial supplier that were determined to be seronegative for Bordetella p68 (ELISA endpoint titer≦1:200). No animals received any vaccines containing antigens against B. bronchiseptica prior to study inclusion. All animals were identified by unique ear tattoos.

Management. Animals were housed individually in runs within one room. Animals were fed according to facility SOPs and water was provided ad libitum.

Allotment. The study design was a completely random design. Animal was the experimental unit.

Masking. Personnel performing serological testing were unaware of individual animal's treatments.

Study Design. All animals were vaccinated subcutaneously with the appropriate vaccine in the intrascapular space on Day 0 and on Day 21.

| Treatment | Investigational Veterinary Product | Animals |
|---|---|---|
| T01 | 15 mcg/dose p68 (new process) with 50 mcg QAC | 5 |
| T02 | 15 mcg/dose p68 (old process) with 50 mcg QAC | 5 |

Vaccines. The experimental vaccines used in this study are briefly described below: All vaccines were stored at refrigerator temperatures. Satisfactory sterility was demonstrated on each experimental vaccine.

1. 15 mcg p68 antigen prepared with newly processed antigen according to the process of the current invention (Example 1), combined with 50 mcg QuilA and 50 mcg cholesterol (experimental serial #59433-81), brought to volume in Hal's buffer.

2. 15 mcg p68 antigen prepared with previously processed antigen according to the process used prior to the current invention, combined with 50 mcg QuilA and 50 mcg cholesterol (experimental serial #59433-9), brought to volume in Hal's buffer.

Challenge. No challenge was administered in this study.

Pertinent Variables Measured.

Blood Sampling. All animals had blood samples collected (approximately 8-10 mls) Day 0, Day 14, Day 21, and Day 35. Samples from all animals on all days were tested by Bordetella p68 specific ELISA and results were reported as reciprocal endpoint titers.

Assessment Of Efficacy.

Criteria for a valid test. All dogs must have initially been seronegative (ELISA endpoint titer≦1:200) against p68. All dogs must have become seropositive (ELISA endpoint titer>1:200) against p68.

Outcome Criteria. The study would be considered successful if all dogs became seropositive against p68, and both groups had similar geometric mean titers.

Data Summary And Analysis. It was determined for each animal whether it was seropositive or seronegative for the p68 antigen on each day of study sample collection beginning with Day 0. Frequency distributions of seropositive/seronegative were calculated for each treatment on each sample day.

Descriptive statistics of p68 titers were calculated for each treatment and day of study including the geometric mean, number of samples, minimum, maximum and 95% CI of the geometric mean.

Results.

Serologic Response to Vaccination. On Day 0 all animals tested seronegative (ELISA endpoint titer≦1:200) for antibodies against Bordetella p68. On Day 14, Day 21, and Day 35 all animals, except one animal in T02 on Day 21, tested seropositive (ELISA endpoint titer>1:200) for antibodies against Bordetella p68. Bordetella p68 endpoint titers are summarized by treatment group in Table 4. Titrations for all Days were started at 50. Any value reported as “less than” was divided by 2 prior to analysis.

TABLE 4
Geometric means of Bordetella p68 endpoint titers

| Day of | | | Geometric | | |
|---|---|---|---|---|---|
| Study | Treatment | | Mean | Minimum | Maximum |
| 0 | T01 | New process | 37.9 | 25 | 50 |
| | T02 | Old process | 50.0 | 25 | 200 |
| 14 | T01 | New process | 4850.3 | 1600 | 12800 |
| | T02 | Old process | 1600.0 | 400 | 12800 |
| 21 | T01 | New process | 1600.0 | 800 | 6400 |
| | T02 | Old process | 800.0 | 100 | 3200 |
| 35 | T01 | New process | 38802.3 | 12800 | 204800 |
| | T02 | Old process | 2425.1 | 800 | 6400 |

Differences between T01 and T02 geometric mean antibody titers following first vaccination, Days 14 and 21, were approximately two- and three-fold, respectively. Geometric mean p68 antibody titers following second vaccination (Day 35) were approximately 16-fold higher in T01 (new process).

Adverse Reactions. No adverse reactions attributed to the vaccination procedure were recorded on either vaccination date.

Discussion.

This study demonstrated that the vaccine formulated with p68 processed by the new method was, if anything, immunologically superior to vaccine formulated with the same amount of p68 antigen processed using the old method. Robust serologic response is desirable from any adjuvanted sub-unit vaccine.

Importantly, the new process by which p68 antigen is produced is a more manufacturing-friendly process. It also produced a more robust immunological response.

Conclusion.

This study fulfilled the criteria that all animals become seropositive (ELISA endpoint titer>1:200) for antibodies against Bordetella p68 following vaccination. There was an approximate 16-fold difference in geometric mean titers between treatment groups following second vaccination (Day 35), so both groups did not have similar geometric mean titers, and therefore, the study did not meet the stated objective. However, the vaccine formulated with p68 antigen processed using the new method elicited a substantially greater serologic response than vaccine formulated with p68 produced using the older method. The new process results in an immunologically superior antigen.

### Example 4

A new canine Bordetella vaccine comprises the p68 outer membrane protein of Bordetella bronchiseptica expressed by Escherichia coli strain LWP68. This protein has demonstrated efficacy in previous experiments. The purpose of this study was to demonstrate the immunogenicity of p68 in minimum-age, susceptible dogs when administered at 15 μg and 45 μg doses with challenge within one month following the last vaccination.

Materials And Methods.

Animals. Forty-five mixed-breed dogs that were 9±1 weeks of age on Day 0 and of mixed sex arrived at the study location on Day-7. Animals were selected from a pool of acceptable animals provided by a commercial supplier, which were determined free from exposure to B. bronchiseptica on Day-21 via negative tracheal swab culture and serum agglutination titers≦1:16 on serological assay for B. bronchiseptica. No animals received any vaccines containing antigens against B. bronchiseptica prior to study inclusion. All animals were identified by unique ear tattoos.

Management. Animals were housed in a barrier facility from birth to the time they were shipped to the study location. During the vaccination phase, animals were group-housed in an isolation building in nine rooms with five animals per room. During the challenge phase, animals were single housed in three rooms with 15 dogs per room.

Allotment. For both the vaccination and challenge phases, animals were randomized in a generalized block design with a room being a block. Animal was the experimental unit.

Masking. Individuals making animal observations and performing serological and microbiological testing were unaware of animals' treatments.

Study Design. All animals were vaccinated subcutaneously with the appropriate vaccine or placebo over the left thoracic wall on Day 0 and over the right thoracic wall on Day 21. The study design is presented below, where abbreviations are the following: Tx=Treatment Group; IVP=Investigational Veterinary Product; SC=Subcutaneous; CFU=Colony Forming Units, BC=Bihr Cat, mcg=microgram, mL=milliliter, and QAC=QuilA Cholesterol.

| | | Dose | | | Total | | Animals |
|---|---|---|---|---|---|---|---|
| Tx | IVP | Volume | Regimen | Animals | Doses | Challenge | Challenged |
| T01 | Saline | 1 mL | 2 Doses, | 15 | 30 | B. bronchiseptica | 15 |
| | | | SC | | | BC strain | |
| | | | | | | ˜1 × 10⁹ CFU | |
| T02 | 15 mcg p68 | 1 mL | 2 Doses, | 15 | 30 | B. bronchiseptica | 15 |
| | in 50 mcg | | SC | | | BC strain | |
| | QAC | | | | | ˜1 × 10⁹ CFU | |
| T03 | 45 mcg p68 | 1 mL | 2 Doses, | 15 | 30 | B. bronchiseptica | 15 |
| | in 50 mcg | | SC | | | BC strain | |
| | QAC | | | | | ˜1 × 10⁹ CFU | |

Vaccines. The experimental vaccines and placebo used in this study are briefly described below. All vaccines were stored at refrigerator temperatures. Satisfactory sterility was demonstrated on each experimental vaccine.
1. Saline—Commercially available 0.9% sterile NaCl solution (AmTech Lot # 304118F Exp. 4/06).
2. 15 mcg p68 antigen prepared according to the process of Example 1 above, combined with 50 mcg QuilA and 50 mcg cholesterol, and brought to volume in phosphate-buffered saline (Serial number: 91703A).
3. 45 mcg p68 antigen prepared according to the process of Example 1 above, combined with 50 mcg QuilA and 50 mcg cholesterol, and brought to volume in phosphate-buffered saline (Serial number: 91703C).

Challenge. The challenge material was prepared according to the following procedure. One vial of B. bronchiseptica (Lot # 051397-85B-2) was diluted 1:100 in Bordetella saline. Three to four Bordet-Gengou agar plates (Lot # 3357049) were streaked with one loopful each, then covered with parafilm and incubated for 48±2 hours at 37.5±2.5° C. Two phase I colonies were selected and streaked per plate on 12 Bordet-Gengou agar plates (Lot # 3357049) and incubated 24±4 hours at 37.5±2.5° C. After incubation, four mls of Bordetella saline (Lot # 0400905) per plate was used to wash colonies from the agar, and the antigen was diluted to an optical density of 0.732 at 600 nm. Approximately 1.8 ml of the challenge material was administered over four minutes via a DeVilbiss nebulizer attached to a nose cone, to each animal after sedation with Ketamine and Xylazine. 0.1 ml aliquots of the challenge material, pre- and post-challenge, were plated onto duplicate Bordet-Gengou agar plates (Lot # 3357049) at 10⁻⁵, 10⁻⁶, and 10⁻⁷ dilutions and incubated at 37.5±2.5° C. for 48±4 hours. Plates with 30-300 colonies were counted and the average concentration was calculated to be 9.35×10⁸ CFU/ml pre-challenge and 1.27×10⁹ CFU/ml post-challenge.

Pertinent Variables Measured.

Tracheal Swabs. All animals had tracheal swabs collected while under sedation on Day-21, Day-5, and Day 45.

All tracheal swabs were streaked on Bordetella specific agar and grown at 37° C.+/−5° C. for 48 hours. Plates were then visually examined for B. bronchiseptica growth, and results were recorded as negative or positive. Those colonies that required further identification were analyzed using the API 20NE (bioMerieux) identification system in conjunction with classical media.

Blood Samples. All animals had blood samples (one 5-6 ml serum separator tube) collected on Day-21, Day-5, Day 0, Day 21, Day 45, and Day 59.

Samples from Days-21,-5, and 45 were analyzed by Bordetella serum agglutination assay. Serial dilutions of each sample were mixed with standardized B. bronchiseptica antigen in 96-well microtiter plates. The mixture was incubated at 37° C. for 2 hours followed by 4° C. for 20 hours. Titers were recorded as the highest dilution of serum that resulted in observable agglutination.

Samples from Day 0, 21, 45, and 59 were analyzed for seroconversion by specific p68 ELISA. Serial dilutions of each sample were mixed and placed in Immulon microtiter plates that had p68 antigen bound. The detecting antibody was enzyme conjugated goat anti-canine IgG. Chromagen substrate was used to detect the antigen-antibody complex and ODs were captured via ELISA reader. Endpoint titers were determined and recorded based on standardized positive control sera.

Pre-vaccination observations. Animals were examined prior to vaccination on Day 0 and Day 21. Rectal temperatures, systemic and local scores, and any pathologic changes noted were recorded.

Post-Challenge Observations. Animals were observed in groups twice daily, once in the morning and once in the afternoon, for between 15-40 minutes from day 46 to day 59. Animals were observed for coughing and any other signs of disease.

Assessment Of Efficacy.

Criteria for a valid test. Animals were to be culture-negative on tracheal swabs and sero-negative on serum agglutination assay both prior to 1^{st} vaccination and prior to challenge. If an animal was positive for either parameter, data from that animal may have been excluded from analysis at the discretion of the Research Investigator.

Outcome Criteria. The study was to be considered successful and a positive test of vaccine immunogenicity if there was a significant (α=0.05) difference in mean percent observation periods an animal was observed coughing between vaccinates and controls.

Data Summary And Analysis.

All results were summarized and statistically evaluated by Pfizer Veterinary Medicine Biometrics, Technology and Quality. The 5% (P≦0.05, two-sided) level of significance was used to measure statistical differences.

Coughing. The percentage of observation periods during which an animal coughed was calculated for each animal. The minimum and maximum percent observation periods coughed were calculated for each treatment. Prior to statistical analysis the data was transformed using the arcsine of the square root of the percentage divided by 100. The transformed percentage was analysed using a general linear mixed model. Pairwise treatment comparisons were made between T01 and treatments T02 and T03. The treatment least squares means were back-transformed to the original scale. The frequency of animals that coughed two and three consecutive observation periods was also calculated for each treatment.

The mixed linear model that was used to analyze percentage times coughing is:
γᵢⱼₖₗ=μ+ρᵢ+βⱼ₍ᵢ₎+τₖ+εᵢⱼₖₗ,
where
γijkl=transformed observation of the i^{th} room, the j^{th} block within the i^{th} room, the k^{th} treatment and the l^{th} animal within the i^{th} room, the j^{th} block and the k^{th} treatment,
μ=overall constant,
ρᵢ random effect of the i^{th} challenge room,
βⱼ₍ᵢ₎=random effect of the j^{th} block (vaccination room) within the i^{th} room,
τₖ=fixed effect of the k^{th} treatment,
εᵢⱼₖₗ=residual.
   (i=1 . . . number of rooms,
   j=1 . . . number of blocks within room,
   k=1 . . . number of treatments,
   l=1 . . . . number of animals within block, treatment and room.)

Titers. Prior to statistical analysis the titers were transformed by taking the logarithm base 2 of the titer. The transformed titers were analysed using a general linear repeated measures mixed model. Pairwise treatment comparisons were made between treatment T01 and treatments T02 and T03 at each day of collection. The treatment least squares means for each day of collection were back-transformed to the geometric means. The minimum and maximum were also calculated for each treatment at each day of collection.

The mixed linear model that was used to analyze the transformed titers is:
γᵢⱼₖₗₘ=μ+ρᵢ+βⱼ₍ᵢ₎+τₖ+αₗ₍ᵢⱼₖ₎+δₘ+τδₖₘ+εᵢⱼₖₗₘ,
where
γᵢⱼₖₗₘ=transformed observation of the i^{th} room, the j^{th} block within the i^{th} room, the k^{th} treatment, the l^{th} animal within the i^{th} room, the j^{th} block and the k^{th} treatment, and the m^{th} timepoint,
μ=overall constant,
ρᵢ=random effect of the ith challenge room,
βⱼ₍ᵢ₎=random effect of the j^{th} block (vaccination room) within the i^{th} room,
τₖ=fixed effect of the k^{th} treatment,
αₗ₍ᵢⱼₖ₎=random effect of the l^{th} animal within the ith room, the j^{th} block and the k^{th} treatment,
δₘ=fixed effect of the m^{th} timepoint,
τδₖₘ=fixed interaction effect of the k^{th} treatment and the m^{th} timepoint,
εᵢⱼₖₗₘ=residual.
   (i=1 . . . number of rooms,
   j=1 . . . number of blocks within room,
   k=1 . . . number of treatments,
   l=1 . . . number of animals within block, treatment and room (or batch),
   m=1 . . . number of timepoints.)
      Results.

Bacteriologic And Serologic Screening. All animals remained free from detectable exposure to b. bronchiseptica until the day of challenge. All animals screened negative on days-21, -5, and 45 by tracheal swab as well as serum agglutination assay with a negative titer being defined as ≦1:16.

Serologic Response To Vaccination. Geometric means of p68 ELISA endpoint titers in dogs from days 0, 21, 45, and 59, following p68 Bordetella vaccination on Days 0 and 21 are summarized in Table 5. Titrations for all Days were started at 50. Any value reported as “less than” was divided by 2 prior to analysis.

TABLE 5
Geometric means of p68 ELISA endpoint titers

Day of Study

| | | | | |
|---|---|---|---|---|
| Treatment | 0 | 21 | 45 | 59 |

| | | | | | |
|---|---|---|---|---|---|
| T01 | Saline | 57 | 87 | 72 | 219 |
| T02 | 15 μg p68 | 72 | 2425 | 117627 | 246380 |
| T03 | 45 μg p68 | 63 | 4032 | 186721 | 204800 |

Significant differences for post vaccination p68 ELISA endpoint titers are listed in Table 6. No significant differences were observed between treatment groups prior to vaccination (Day 0). Only significant (P≦0.05) contrasts are shown.

TABLE 6
Treatment group comparisons

| Day of Study | Contrast | P-value |
|---|---|---|
| 21 | T01 vs T02 | ≦0.0001 |
| | T01 vs T03 | ≦0.0001 |
| 45 | T01 vs T02 | ≦0.0001 |
| | T01 vs T03 | ≦0.0001 |
| 59 | T01 vs T02 | ≦0.0001 |
| | T01 vs T03 | ≦0.0001 |

Post-challenge observations. Animals were observed for coughing by qualified individuals twice daily for 14 days following challenge procedures. The animals were recorded as either Y (observed coughing) or N (not observed coughing) and any other signs of disease were recorded as comments.

Presented in Table 7 are mean, minimum, and maximum percent observation periods coughing was observed in unvaccinated and p68 Bordetella vaccinated dogs following aerosol challenge with B. bronchiseptica.

TABLE 7
Percent observation periods coughing

| | | Number of | | | |
|---|---|---|---|---|---|
| Treatment | | Animals | Mean | Minimum | Maximum |
| T01 | Saline | 15 | 45.9% | 0.0% | 85.7% |
| T02 | 15 μg p68 | 15 | 4.7% | 0.0% | 17.9% |
| T03 | 45 μg p68 | 15 | 10.3% | 0.0% | 42.9% |

Significant differences of treatment comparisons for percent observation periods coughed between unvaccinated and p68 Bordetella vaccinated animals following aerosol challenge with B. bronchiseptica are listed in Table 8. Only significant (P≦0.05) contrasts are shown. A significant difference was found between T01 (saline) and T02 (15 μg p68) as well as between T01 (saline) and T03 (45 μg p68).

TABLE 8
Treatment comparisons - observation periods coughed

| Contrast | P-value |
|---|---|
| T01 vs T02 | ≦0.0001 |
| T01 vs T03 | 0.0004 |

The frequencies of animals in each treatment group that coughed two and three consecutive observation periods are listed in Table 9.

TABLE 9
Consecutive observation periods coughed

| | | | |
|---|---|---|---|
| | Number of | Coughed two | Coughed three |
| Treatment | Animals | consecutive periods | consecutive periods |

| | | | | |
|---|---|---|---|---|
| T01 | Saline | 15 | 14 (93%) | 13 (87%) |
| T02 | 15 μg p68 | 15 | 1 (7%) | 0 (0%) |
| T03 | 45 μg p68 | 15 | 5 (33%) | 3 (20%) |

Adverse Reactions. No adverse reactions that were attributed to the vaccination procedure were recorded for either vaccine dose at either vaccination date.

Discussion.

This study successfully demonstrates that the vaccine is protective against virulent challenge at both the minimum immunizing dose, and at 45 μg p68/dose, when administered to dogs at 9 and 12±1 weeks of age.

Animals were screened to ensure that they had not been exposed to B. bronchiseptica prior to challenge. Exceeding care had to be taken to protect the animals from exposure to this ubiquitous organism. The challenge material, a strain of B. bronchiseptica historically used to challenge animals due to it's consistent ability to cause clinical signs in susceptible animals, proved to be highly virulent again in this study. Thirteen of the fifteen animals in the saline-vaccinated group of animals displayed significant clinical signs. These signs included not only coughing but also the retching, productive spasmodic episodes which are classically associated with Infectious Tracheobronchitis. There is no doubt that this was an adequate challenge.

Coughing in B. bronchiseptica models have been analyzed in several ways. Percent observations coughing is the most widely accepted, however, some prefer an analysis which classifies animals as responders and non-responders. Depending on the precedent, responders are defined as animals with two or three consecutive observation periods in which they are observed coughing. It is worth discussion that in this study, there is only one responder (by either definition) in group T02, and only five responders, using the two consecutive periods criteria, in group T03, while there are 14 responders, using the two consecutive periods criteria, in group T01. By any definition, the vaccine protected the animals from the virulent challenge.

Conclusion.

This study is a successful reference qualification of the 15 μg p68/dose vaccine. It also establishes the 45 μg p68/dose vaccine as protective against challenge.

### Example 5

This study was designed to determine if the combination vaccine of Vanguard® 5rB [Canine Distemper-Adenovirus Type 2-Parainfluenza-Parvovirus, Modified Live Virus-Bordetella Bronchiseptica Bacterial Extract, Subunit (USDA Product Code 46E9.R0)], administered to young animals, results in elevated p68 antibody titers. Antibody titers in these animals were compared with those in animals vaccinated with saline plus the modified-live components of the vaccine [Vanguard® Plus 5-Canine Distemper-Adenovirus Type 2-Parainfluenza-Parvovirus, Modified Live Virus (USDA Product Code 13D1.22)]. Elevated levels of p68 antibody titers would indicate that there was no interference from the modified-live components of the vaccine composition.

Materials and Methods.

Animals. Thirty beagle dogs of either sex were used in this study. Animals were 6±1 weeks of age at the time of first vaccination. Animals were seronegative for antibodies against Canine Distemper Virus (CDV), Canine Adenovirus Type 2 (CAV-2), Canine Parainfluenza (CPI), Canine Parvovirus (CPV), and Bordetella p68 prior to first vaccination. Animals were determined to be free from exposure to Bordetella bronchiseptica via tracheal swab culture and serum agglutination assay prior to the first vaccination. Animals did not receive any vaccines containing CDV, CAV-2, CPI, CPV, or Bordetella antigens prior to Day 0.

Animal Management. Animals were group housed within five rooms with six animals per room. All animals had access to age-appropriate food and water. Animals were observed by qualified personnel at least daily for morbidity or mortality.

Allotment. Animals were randomly allocated to treatments, rooms, and pens.

Masking. All personnel making animal observations were unaware of individual treatments. Non-screening serum samples were labeled in such a way as to make laboratory personnel unaware of treatment group or animal of origin.

Study Design

| | | Dose | Reg- | Ani- | Total |
|---|---|---|---|---|---|
| Tx | IVP | Volume | imen | mals | Doses |
| T01 | Canine Distemper-Adenovirus | 1 mL | 3 Doses | 15 | 45 |
| | Type 2-Parainfluenza- | | SC | | |
| | Parvovirus Vaccine, Modified | | | | |
| | Live Virus [USDA Product | | | | |
| | Code 13D1.22] | | | | |
| | reconstituted with sterile | | | | |
| | water | | | | |
| T02 | Canine Distemper-Adenovirus | 1 mL | 3 Doses | 15 | 45 |
| | Type 2-Parainfluenza- | | SC | | |
| | Parvovirus Vaccine, Modified | | | | |
| | Live Virus-Bordetella | | | | |
| | Bronchiseptica Bacterial | | | | |
| | Extract, Subunit [USDA | | | | |
| | Product Code 46E9.R0 | | | | |
| | (combination of USDA | | | | |
| | Product Codes 13D1.22 | | | | |
| | and 2B05.R0)] | | | | |

Abbreviations:
Tx = Treatment Group;
IVP = Investigational Veterinary Product;
SC = Subcutaneous;
mL = milliliter.

Vaccines. The experimental vaccines and placebo used in this study are briefly described below. All vaccines were stored at refrigerator temperatures.

1. Vanguard® Plus 5 [Canine Distemper-Adenovirus Type 2-Parainfluenza-Parvovirus Vaccine, Modified Live Virus, USDA Product Code 13D1.22], all antigens at ≧release levels (Serial number: A365332B).

2. Bordetella Bronchiseptica Bacterial Extract, Subunit, 45 mcg p68 antigen prepared according to Example 1 above, combined with 50 mcg QuilA and 50 mcg cholesterol, and brought to volume in phosphate-buffered saline (Serial number: 91703C).

Procedures.

Prior to vaccination, animals were examined and a blood sample (one 6-8 ml serum separator tube [SST]) was collected. Potential injection sites were examined. Local and systemic scores, and any pathologic changes were noted. Animals were vaccinated subcutaneously in the intrascapular space with the appropriate vaccine according to the allotment. Animals were observed (as a group) for approximately 20 minutes following vaccination for any adverse events.

Day 62±3 days. Animals were examined and a blood sample (one 6-8 ml SST) and tracheal swabs were collected.

Assay of Specimens. Serum samples were spun, and the serum was decanted into labeled individual plastic cryovials.

Serum samples collected for screening purposes prior to Day 0, were analyzed for exposure to B. bronchiseptica by serum agglutination assay. Briefly, serial dilutions of candidate sera were mixed with standardized B. bronchiseptica antigen in 96 well microtiter plates. The mixture was incubated for 2 hours at 37° C. followed by 4° C. for 20 hours. Titers were reported as the highest dilution of serum that resulted in observable agglutination.

Tracheal swabs collected prior to and on Day 0 and on Day 62+/−3 days were streaked on Bordetella specific agar and grown at 37° C.+/−5° C. for 48 hours. Plates were then visually examined for growth. If identification was necessary, the API 20NE (bioMerieux) identification system in conjunction with classical media was used.

Serum samples collected on Day 0 and Day 62 were tested by specific Bordetella p68 ELISA and serum neutralization assays for CDV, CAV-2, CPI, and CPV.

Outcome Criteria. Non-interference of CDV, CAV-2, CPI, and CPV with Bordetella p68 would be demonstrated, if on day 62, the animals in the T02 treatment exhibited significantly elevated p68 antibody titers when compared with the p68 antibody titers in the T01 treatment group.

Results.

Serologic Response To Vaccination. No Bordetella bacteria were isolated from tracheal swabs taken from animals on day 0 and day 62. On Day 0, all animals tested seronegative (ELISA endpoint titer≦1:200) for antibodies against Bordetella p68. On Day 62, all animals except two in the T01 group tested seropositive (ELISA endpoint titer>1:200) for antibodies against Bordetella p68. The two animals had endpoint titers equal to 200. Bordetella p68 endpoint titers are summarized by treatment group in Table 10. Titrations for all samples were started at 50. Any value reported as “less than” was divided by 2 prior to analysis. The mean p68 antibody titer for treatment group T02 was more than 50 times higher than the mean p68 antibody titer for treatment group T01 on study Day 62.

TABLE 10
Bordetella p68 endpoint titers

| Day of | | | Geometric | | |
|---|---|---|---|---|---|
| Study | Treatment | | Mean | Minimum | Maximum |
| 0 | T01 | Vanguard ® Plus 5 | 67 | 25 | 200 |
| | T02 | Vanguard ® 5 r B | 61 | 25 | 200 |
| 62 | T01 | Vanguard ® Plus 5 | 672 | 200 | 1600 |
| | T02 | Vanguard ® 5 r B | 36204 | 6400 | 204800 |

Treatment comparisons for p68 ELISA endpoint titers are listed in Table 11. There was no significant difference between treatment groups in p68 antibody titers at Day 0. The p68 antibody titers differed significantly (p≦0.05) between treatment groups at Day 62.

TABLE 11
Treatment Group Comparisons

| Day of Study | Contrast | P-value |
|---|---|---|
| 0 | T01 vs T02 | 0.6864 (ns) |
| 62 | T01 vs T02 | ≦0.0001 |

Discussion.

In this study, animals that received a dose of 45 mcg of p68 in the Vanguard® 5rB combination vaccine (Canine Distemper-Adenovirus Type 2-Parainfluenza-Parvovirus, Modified Live Virus-Bordetella Bronchiseptica Bacterial Extract Subunit), prepared according to the process of Example 1, had a mean p68 antibody titer that was more than 50 times higher (P≦0.0001) than animals that received only the modified-live component (the Vanguard® Plus 5 vaccine—Canine Distemper-Adenovirus Type 2-Parainfluenza-Parvovirus, Modified Live Virus). Such a rise in antibody levels as seen in the group that received the Vanguard® 5rB combination vaccine was also seen in prior studies of monovalent vaccine compositions prepared according to the process of Example 1, (see Examples 3 and 4). This indicates that the modified-live component of the composition did not interfere with the production of p68 antibodies.

The p68 vaccine composition formulated according to the process of Example 1, elicited a substantially greater serologic response than that seen in a prior study (Example 3) of a p68 vaccine formulated with antigen composition prepared with about 0.1% SDS (w/v). The vaccine composition containing antigen composition prepared with about 0.003% SDS is an immunologically superior vaccine compared with the vaccine composition formulated with 0.1% SDS antigen composition.

This study successfully demonstrated that the Vanguard® 5rB combination vaccine caused significant elevation of p68 antibody titers in young dogs of approximately 6 weeks of age.

Exceeding care was taken to protect the animals from exposure to B. bronchiseptica before and after vaccination. In addition, animals were screened to ensure that they had not been exposed to B. bronchiseptica prior to vaccination. No Bordetella bacteria were isolated from tracheal swabs taken from animals on Day 0 and Day 62, but a slight elevation in the antibody titers for the T01 group on Day 62 indicates that the animals may have had some exposure to the bacterium by Day 62.

### CONCLUSIONS

The Vanguard® 5rB vaccine combines the efficacy and safety of the Bordetella Bronchiseptica Bacterial Extract, Subunit (p68) vaccine against Bordetella bronchiseptica with the proven performance of Vanguard® Plus 5 modified-live virus vaccine against Canine Distemper Virus (CDV), Canine Adenovirus Type 2 (CAV-2), Canine Parvovirus (CPV), and Canine Parainfluenzavirus (CPI).

The present invention has been described in detail and by reference to various specific and preferred embodiments and techniques. However, it should be understood that many variations and modifications can be made while remaining within the scope of the invention.

What is claimed is:

1. An antigen composition comprising a therapeutically effective amount of p68 protein and an amount of sodium dodecyl sulfate, wherein the amount of sodium dodecyl sulfate is from about 0.0005 percent to about 0.08 percent (w/v).

2. The composition of claim 1, wherein the amount of sodium dodecyl sulfate is from about 0.001 percent to about 0.01 percent (w/v).

3. The composition of claim 1, wherein the amount of sodium dodecyl sulfate is from about 0.0025 percent to about 0.0035 percent (w/v).

4. The composition of claim 1, wherein the p68 protein comprises a polypeptide selected from the group consisting of
a) an amino acid sequence set forth in SEQ ID NO: 1; and
b) an amino acid sequence that has at least 90% sequence identity and/or homology to the amino acid sequence set forth in SEQ ID NO: 1.

5. The composition of claim 1, wherein the p68 protein is produced from a polynucleotide sequence that encodes a p68 protein comprising an amino acid sequence set forth in SEQ ID NO: 1, or an amino acid sequence that has at least 90% sequence identity and/or homology to the amino acid sequence set forth in SEQ ID NO: 1.

6. The composition of claim 5, wherein the polynucleotide sequence has a sequence of SEQ ID NO: 2, or a polynucleotide sequence that has at least 90% sequence identity and/or homology to the polynucleotide sequence set forth in SEQ ID NO: 2.

7. The composition of claim 1, wherein the amount of p68 protein is about 2 to about 100 μg per dose.

8. The composition of claim 1, wherein the amount of p68 protein is about 4 to about 45 μg per dose.

9. The composition of claim 1, wherein the composition has a pH from about 9.5 to about 13.

10. The composition of claim 1, wherein the composition has a pH from about 10 to about 12.

11. A vaccine composition comprising the antigen composition of claim 1, and further comprising a carrier.

12. The vaccine composition of claim 11, wherein the carrier comprises saponin as a surfactant.

13. The vaccine composition of claim 12, wherein the saponin is Quil A as the surfactant combined with cholesterol.

14. The vaccine composition of claim 13, wherein the amount of Quil A is about 1 to about 100 μg per dose, and the amount of cholesterol is about 1 to about 100 μg per dose.

15. The vaccine composition of claim 13, wherein the amount of Quil A is about 10 to about 50 μg per dose, and the amount of cholesterol is about 10 to about 50 μg per dose.

16. The vaccine composition of claim 11, wherein the carrier comprises aluminum hydroxide.

17. The vaccine composition of claim 11, wherein the composition has a pH from about 6 to about 9.

18. The vaccine composition of claim 11, wherein the composition has a pH from about 6.5 to about 8.0.

19. A method of protecting a canine from infection comprising the step of administering to the canine a therapeutically effective amount of the composition of claim 11.

20. The vaccine composition of claim 11, further comprising one or more antigens selected from the group consisting of canine distemper (CD) virus, canine adenovirus type 1 (CAV-1), canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, Leptospira pomona, Leptospira hardjobovis, and Leptospira hardjo.

21. A method of protecting a canine from infection comprising the step of administering to the canine a therapeutically effective amount of the composition of claim 20.

22. The vaccine composition of claim 11, wherein the amount of p68 protein is about 4 to 45 μg per dose; the carrier is Quil A in an amount of about 10 to about 50 μg per dose and cholesterol in an amount of about 10 to about 50 μg per dose; the composition has a pH from about 6.5 to about 8.0; and the composition further comprises antigens of canine distemper (CD) virus, canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, and Leptospira pomona.

23. A method of protecting a canine from infection comprising the step of administering to the canine a therapeutically effective amount of the composition of claim 22.

24. A process for producing an antigen composition comprising the steps of
a) suspending inclusion bodies containing p68 protein in a buffer solution having a pH from about 9.5 to about 13; and
b) adding sodium dodecyl sulfate to a concentration of about 0.0005 percent to about 0.08 percent (w/v).

25. The process of claim 24, wherein the amount of sodium dodecyl sulfate is from about 0.001 percent to about 0.01 percent (w/v).

26. The process of claim 24, wherein the amount of sodium dodecyl sulfate is from about 0.0025 percent to about 0.0035 percent (w/v).

27. The process of claim 24, wherein the p68 protein comprises a polypeptide selected from the group consisting of
a) an amino acid sequence set forth in SEQ ID NO: 1; and
b) an amino acid sequence that has at least 90% sequence identity and/or homology to the amino acid sequence set forth in SEQ ID NO: 1.

28. The process of claim 24, wherein the amount of p68 protein is about 2 to about 100 μg per dose.

29. The process of claim 24 wherein the amount of p68 protein is about 4 to about 45 μg per dose.

30. The process of claim 24, wherein the buffer solution is a carbonate buffer.

31. The process of claim 24, further comprising a step of clarifying the composition by filtration or centrifugation.

32. The process of claim 24, further comprising a step of sterilizing the composition.

33. The process of claim 32, wherein the composition is sterilized by filtration.

34. The process of claim 24, wherein the p68 protein is produced by steps comprising
a) cloning into an expression vector a polynucleotide sequence that encodes a p68 protein comprising an amino acid sequence set forth in SEQ ID NO: 1, or an amino acid sequence that has at least 90% sequence identity and/or homology to the amino acid sequence set forth in SEQ ID NO: 1;
b) introducing the expression vector into a bacterial cell; and
c) expressing the p68 protein, which accumulates in inclusion bodies.

35. The process of claim 34, wherein the polynucleotide sequence has a sequence of SEQ ID NO: 2, or a polynucleotide sequence that has at least 90% sequence identity and/or homology to the polynucleotide sequence set forth in SEQ ID NO: 2.

36. The process of claim 34, wherein the bacterial cell is Escherichia coli.

37. The process of claim 24, comprising the additional step, after step b, of combining the antigen composition with a carrier, said carrier having a pH from about 6.5 to about 8.0.

38. The process of claim 37, wherein the carrier is Quil A and cholesterol.

39. A method of protecting a canine from infection comprising the step of administering to the canine a therapeutically effective amount of the composition produced by the process of claim 37.

40. A method of protecting a canine from infection comprising the step of administering to the canine a therapeutically effective amount of the composition produced by the process of claim 37, wherein the p68 protein is produced by steps comprising
a) cloning into an expression vector a polynucleotide sequence that encodes a p68 protein comprising an amino acid sequence set forth in SEQ ID NO: 1, or an amino acid sequence that has at least 90% sequence identity and/or homology to the amino acid sequence set forth in SEQ ID NO: 1.;
b) introducing the expression vector into a bacterial cell; and
c) expressing the p68 protein, which accumulates in inclusion bodies.

41. A method of claim 40, wherein the polynucleotide sequence has a sequence of SEQ ID NO: 2, or a polynucleotide sequence that has at least 90% sequence identity and/or homology to the polynucleotide sequence set forth in SEQ ID NO: 2.

42. The process of claim 37, further comprising a step of adding to the antigen composition one or more antigens selected from the group consisting of canine distemper (CD) virus, canine adenovirus type 2 (CAV-2), canine parainfluenza (CPI) virus, canine coronavirus (CCV), canine parvovirus (CPV), Leptospira bratislava, Leptospira canicola, Leptospira grippotyphosa, Leptospira icterohaemorrhagiae, Leptospira pomona, Leptospira hardjobovis, and Leptospira hardjo.

43. A method of protecting a canine from infection comprising the step of administering to the canine a therapeutically effective amount of the composition produced by the process of claim 42.
